(19) United States
(12) Patent Application Publication
(10) Pub. No.: US 2024/0257957 A1
(43) Pub. Date: Aug. 1, 2024

(54) SYSTEM AND METHOD OF MANAGING PROFESSIONAL SERVICE NETWORK

(71) Applicant: Vanessa Solano, Santa Ana, CA (US)

(72) Inventor: Vanessa Solano, Santa Ana, CA (US)

(21) Appl. No.: 18/425,951

(22) Filed: Jan. 29, 2024

Related U.S. Application Data
(60) Provisional application No. 63/442,169, filed on Jan. 31, 2023.

(51) Int. Cl. G16H 40/20; G16H 10/20

(52) U.S. Cl. CPC G16H 40/20; G16H 10/20

(57) ABSTRACT

A computer-implemented method for managing a network-based service, comprising: establishing a provider portal for a plurality of providers to each create a provider profile; establishing a facility portal for at least one facility to send a vacancy request to fulfill one or more vacancies; receiving, over one or more networks, the vacancy request from the facility; determining a set of potential providers, to fulfill the vacancies, based on the provider profiles including availabilities, a compliance requirement, and the vacancy request, wherein the compliance requirement includes a government compliance requirement and a facility compliance requirement; determining a ranking of the set of the providers based on the provider profiles and the vacancy request; and arranging the providers to fulfill the vacancies based at least in part on the ranking and the provider profiles until all vacancies have been fulfilled.

### TECHNICAL FIELD

The present disclosure relates to system and method of managing a supply and demand of professional service. More specifically, the present disclosure relates to system and method of managing the demands of facilities seeking professional services by inviting professional service providers to supply those demands.

### BACKGROUND

Fueled by factors like employee burnout, an aging population and a dearth of training, states across the country are facing a familiar and common problem: a nursing shortage. The shortage of nurses and clinicians nationwide has a cascading affect for healthcare facilities. Hospitals and health systems continue to find themselves in a desperate hunt for available frontline nurses to relieve their exhausted staff. With continued increases in nurse turnover and staffing issues, reliance on a more mobile workforce continues as hospitalization rates driven by the Covid-19 pandemic continue to waver.

However, for healthcare and hospital administration, it can also be a challenge to stay on top of every individual licensing board across the entire country. At times, it can be difficult enough to verify that the license is valid and active. It can be a manual, time-consuming process, preventing legitimate talent from providing care as quickly as possible. With patient safety as a top priority, the cost of having credentials and license verification tied up in a slow process is the ability to provide care at the moment patients need it.

Credentialing and license monitoring for traveling nurses also have their own set of nuances. In addition to administrative challenges that come with monitoring this mobile workforce, the Nurse Licensure Compact or NLC, is a multi-state agreement that allows nurses to practice in any participating state, and can sometimes be tricky to monitor. Known as one of the most popular and widely supported compacts in the country, it enables nurses to move across the country without obtaining any additional licenses. For nurses moving from a compact state to a non-compact state or moving from a non-compact state to another non-compact state, the process is complicated (to say the least). For this reason, providers should pay close attention when investigating whether their travel nurses have the correct licensure.

It is critical to have a credentialing and ongoing monitoring solution in place that can monitor state license boards. If a traveling nurse is working at a given hospital, that nurse is treating patients on behalf of the provider organization. It's important to add these nurses to your ongoing license and exclusion monitoring environment. It is similarly crucial for a solution to ensure that licenses are active and that both online and offline sources are verified. Ensuring that license and exclusion monitoring is a part of daily compliance and HR workflows can help healthcare organizations easily determine whether their providers are in good standing and eligible to provide safe healthcare to their patients.

Accordingly, there is a need for a system and method of managing the demands of facilities seeking professional services by inviting professional service providers to supply those demands. There is also a need for a system and method of ensuring compliance and credentials of service providers at the facility are always in good standing.

### SUMMARY

The present invention relates to a system and a method for managing a system of professional service providers and facilities that require professional services. The system includes a provider device, a network, a facility device, a server, wherein said devices communicate amongst themselves over the network. In the present embodiment, the server includes one or more processors and one or more electronic memories configured to store computer instructions, wherein the processor executes the instructions to allow professional service providers and facilities to establish portals to accept information which is later used to formulate a plan to invite the providers to fulfill the demand of facilities for professional services. In the present embodiment, the professional service providers are registered nurse (RN) who has graduated or successfully passed a nursing program from a recognized nursing school and met the requirements outlined by a country, state, or similar government-authorized licensing body to obtain a nursing license. On the other hand, facilities, facilities are places that provide health care. They include hospitals, clinics, outpatient care centers, and specialized care centers, such as birthing centers and psychiatric care centers. In different embodiments, the providers can be other professionals such as doctors, dancers, accountants, lawyers, etc. that provide professional services. For brevity, the professional service provider will now simply be collectively called the provider. Similarly, personnel working at the facility will now be collectively called the facility.

The portal is a specially designed website that collect information from and distributes information to the professional service providers and facilities. For professional service providers, portals allow them to update their credentials for compliance, accept invitation to work shift, cancel shifts, give feedback at the end of shifts, etc. For facilities, portals allow them to set up credential or compliance requirements for providers to update and satisfy, creating vacancies of shifts to be fulfilled by providers, choose the providers on a recommendation list to fulfill the vacancies, etc. The present invention allows the facility to create vacancy request and the provider to create shift availability offer even before the existence of a demand for the shift.

In different embodiments, the system and method can generate a ranked list of potential providers for the facility's selection, showing the facility personnel shift availability offer already in place to fulfill the vacancy, sends more invitations to potential providers when not enough providers accept to fulfill the vacancy, accept compliance requirements from the facility, require the providers to satisfy the requirements in order to work at the facility, collect information of the provider's performance, use the information to update the provider's profile for future references, request shift confirmation from the provider to keep time record of the shift for future references, allow the facility to establish criteria for determining whether certain provider should no longer be working at the facility, allow the provider to perform functions such as requesting breaks and keeping time records of the break.

### BRIEF DESCRIPTION OF THE DRAWINGS

Non-limiting and non-exhaustive embodiments of the present disclosure are described with reference to the following figures, wherein like reference numerals refer to like parts throughout the various figures unless otherwise specified.

FIG. 1 illustrates a system for managing a network of professional service provider and facility.

FIG. 2 illustrates an example of the facility portal for a facility to create vacancy request.

FIG. 3a illustrates the process of generating invitation to professional service provider to fulfill a shift vacancy.

FIG. 3b illustrates an alternative embodiment to generate invitation to professional service provider to fulfill a shift vacancy.

FIG. 4 illustrates a provider portal for a provider to create availability offer.

FIG. 5a illustrates the embodiment where the provider cancels the shift that he/she previously agreed to fulfill.

FIG. 5b illustrates an alternative embodiment of the system that generates a ranked list of potential providers for the facility's selection.

FIG. 6 illustrates the facility portal showing a provider with an availability offer for the vacancy that the facility just created.

FIG. 7 illustrates the scenario where the system sends more invitations to potential providers when not enough providers accept to fulfill the vacancy.

FIGS. 8a-b illustrate an alternative embodiment of the system that accept compliance requirements from the facility and requires the providers to satisfy the requirements in order to work at the facility.

FIG. 9a illustrates an alternative embodiment of the system that collect information of the provider's performance and use the information to update the provider's profile for future references.

FIG. 9b illustrates an alternative embodiment of the system that request shift confirmation from the provider to keep record of the begin and end time of shift for future references.

FIG. 10 illustrates an alternative embodiment of the system that allows the facility to establish criteria for determining whether certain provider should no longer be working at the facility.

FIGS. 11a-c illustrates embodiments of the system that allows the provider to perform functions such as requesting breaks and keeping time records of the break.

FIG. 12 illustrates a method of managing a system of professional service providers and facilities that require professional services.

FIG. 13 illustrates an alternative embodiment of the method of arranging professional service providers to fulfill the vacancy at the facility by creating and sending a recommendation list.

FIG. 14 illustrates an embodiment of the method that allows provider to pre-offer his/her availability.

FIG. 15 illustrates the scenario that no one responds or accepts the invitation to fulfill the shift vacancy.

FIG. 16 illustrates another embodiment of the method that allows the facility to collect information regarding the performance of the providers.

FIG. 17 illustrates yet another embodiment of the method that allows the facility to establish criteria that determines whether the provider should no longer be working in the facility.

FIG. 18 illustrates yet another embodiment of the method that allows the facility to make additions such as compliance or credential requirements.

### DETAILED DESCRIPTION

The embodiments can now be better understood by turning to the following detailed description of the embodiments, which are presented as illustrated examples of the embodiment defined in the claims. It is expressly understood that the embodiments as defined by the claims may be broader than the illustrated embodiments described below. Many alterations and modifications may be made by those having ordinary skill in the art without departing from the spirit and scope of the embodiments.

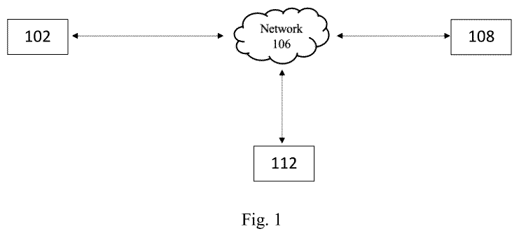

FIG. 1 illustrates a basic example of managing a system of professional service providers and facilities that require professional services. The system includes a provider device 102, a network 106, a facility device 108, a server 112, wherein said devices 102, 106, 112 communicate amongst themselves over the network 106. In the present embodiment, the server 112 includes one or more processors and one or more electronic memories configured to store computer instructions, wherein the processor executes the instructions to allow professional service providers and facilities to establish portals to accept information which is later used to formulate a plan to invite the providers to fulfill the demand of facilities for professional services. In the present embodiment, the professional service providers are registered nurse (RN) who has graduated or successfully passed a nursing program from a recognized nursing school and met the requirements outlined by a country, state, or similar government-authorized licensing body to obtain a nursing license. On the other hand, facilities, facilities are places that provide health care. They include hospitals, clinics, outpatient care centers, and specialized care centers, such as birthing centers and psychiatric care centers. In different embodiments, the providers can be other professionals such as doctors, dancers, accountants, lawyers, etc. that provide professional services. For brevity, the professional service provider will now simply be collectively called the provider. Similarly, personnel working at the facility will now be collectively called the facility.

The portal is a specially designed website that collect information from and distributes information to the professional service providers and facilities. For professional service providers, portals allow them to update their credentials for compliance, accept invitation to work shift, cancel shifts, give feedback at the end of shifts, etc. For facilities, portals allow them to set up credential or compliance requirements for providers to update and satisfy, creating vacancies of shifts to be fulfilled by providers, choose the providers on a recommendation list to fulfill the vacancies, etc. The activities mentioned above that can be done in portals by both providers and facilities will be discussed in more details below.

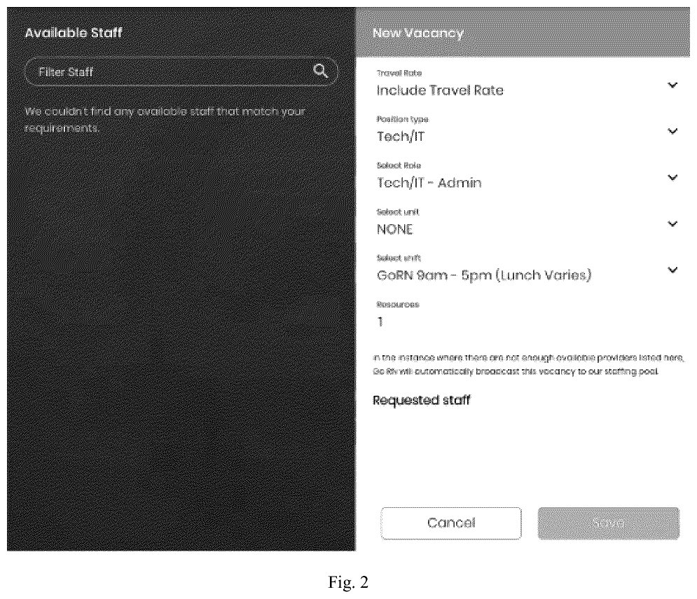

FIG. 2 is an exemplary view of the facility portal for a facility to create vacancy request for the server 112 to use as a beginning point to compile a list of providers to ask to fulfill the vacancy. As illustrated in FIG. 2, the facility portal allows the facility personnel to supply information such as travel rate for travelling nurses, position and role to be fulfilled, and shift time that needs to the provider to be available.

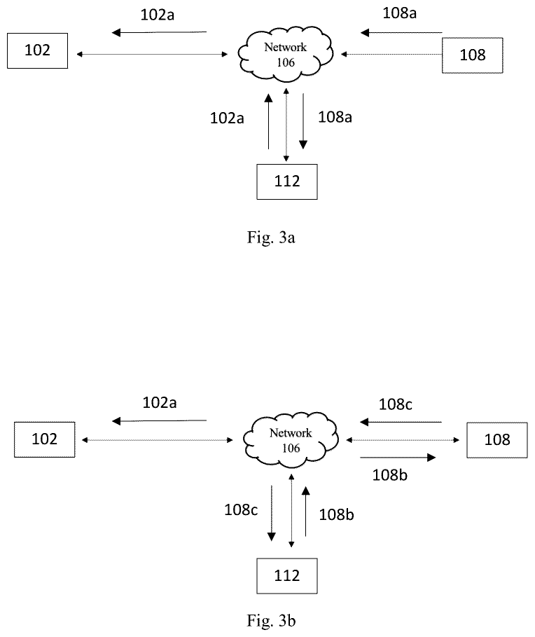

FIG. 3a illustrates the process of generating invitation to professional service provider to fulfill a shift vacancy at a facility. When there is one or more vacancies of shifts to be fulfilled, the facility can use the facility device 108 such as a laptop or desktop computer to create a vacancy request 108a and send it over the network 106 to the server 112. The vacancy request 108a preferably includes information related to the shift in question such as location of the shift, role to be fulfilled, time of the shift etc. Once the server 112 receives the vacancy request 106a, it will review the provider information stored in the database to look for provider that can fulfill the vacancy, based on the information included in the vacancy request 106a plus other information such as compliance status and credentials. The server 112 then determines a list of potential providers to fulfill the vacancy based on information associated with each provider and the vacancy in question. The server 112 then ranks the potential providers based on criteria such as availabilities of the providers, credential, compliance status, etc. in order to find the best fit to fulfill the vacancy. Finally, the server 112 will send an invitation 102a to potential providers for his/her acceptance to fulfill the vacancy. In another embodiment, the server 112 may automatically send out invitation. For instance, the server 112 may automatically send invitations to providers already declared their availabilities before the vacancy is created by the facility. If there is vacancy to be fulfilled, then the server 112 will create another list of potential providers for facility review and send out invitations automatically or upon facility approval, until all vacancies area fulfilled.

In another embodiment illustrated in FIG. 3b, the server 112 first sends the ranked list 108b of providers to facility for selection. The ranked list 108b includes providers' information in the order from the most suitable to the least suitable, based on the information stored in the server 112. The facility 108 can then make determination on choosing which provider to fulfill the shift vacancy and send the selection decision 108c to the server 112. Then the server 112 will send out invitations to the provider based on the decision by the facility.

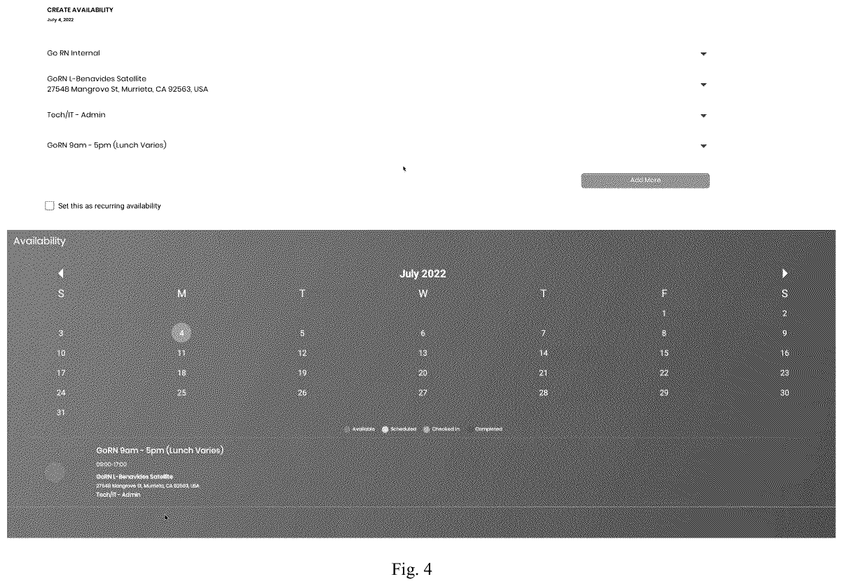

FIG. 4 is an exemplary view of the provider portal for a provider to create availability. As illustrated, the provider can supply information such as the location of the shift he/she is available to work, role of the provider, and shift time that he/she is available. Once the provider has confirmed his/her availability, the information will be sent from the provider device 102 to the server 112 for storage. When the server 112 receives a vacancy request 108a from the facility, it will check its database to see if there is any provider who has already offered available for the shift in question. If there is indeed one or more provider already available, the server 112 may automatically send out invitations 102a to the providers for acceptance to fulfill the vacancy. Alternatively, the server 112 may first send the recommendation list to the facility that includes the provider with availability, wait for the facility's choice/approval, and send invitation to the chosen provider.

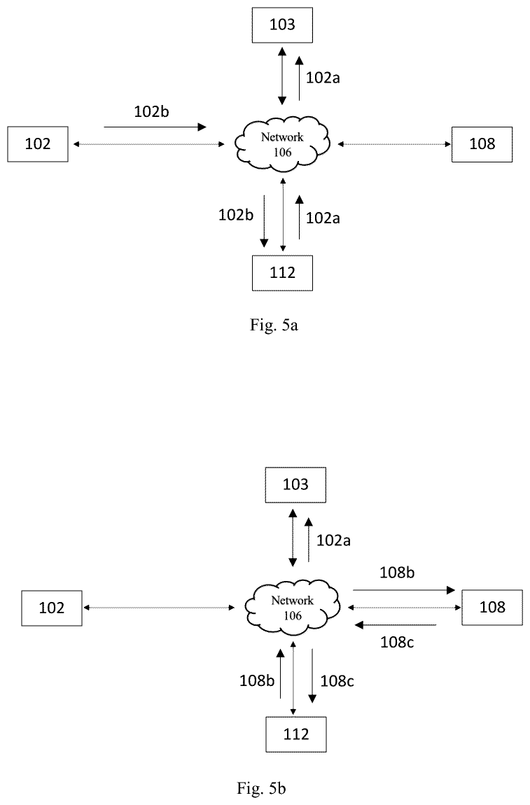

FIG. 5a illustrates the possible scenario where the provider cancels the shift that he/she previously agree to fulfill. Firstly, the provider needs to send a cancellation notification 102b through the provider portal using the provider device 102 such a his/her mobile phone or personal computer. Once the server 112 has confirmed the receipt of the cancellation notification 102b, it will determine a new list of potential providers to fulfill the vacancy based on information in their respective portals and the vacancy in question. The server 112 then ranks the potential providers based on criteria such as availabilities of the providers, credential, compliance status, etc. in order to find the best fit to fulfill the vacancy. Finally, the server 112 will send invitations to potential provider 103 for his/her acceptance to fulfill the vacancy. Basically, the server 112 repeats the same process that got the acceptance of the previous provider 102. The server 112 will also update the shift schedule on the facility portal to make sure that the facility is up to date with the vacancy fulfilling processing. The process described above occurs without the facility's involvement, at least partly to save waiting time to fulfill the new vacancy.

In another embodiment illustrated in FIG. 5b, before sending invitation to new potential provider, the server 112 can again sends the ranked list 108b of providers to facility for selection. The ranked list 108b includes providers' information in the order from the most suitable to the least suitable, based on the information stored in the server 112. The facility 108 can then make determination on choosing which provider to fulfill the shift vacancy and send the selection decision 108c to the server 112. Then the server 112 will send out invitations 102a to the new potential provider 103 based on the decision by the facility 108.

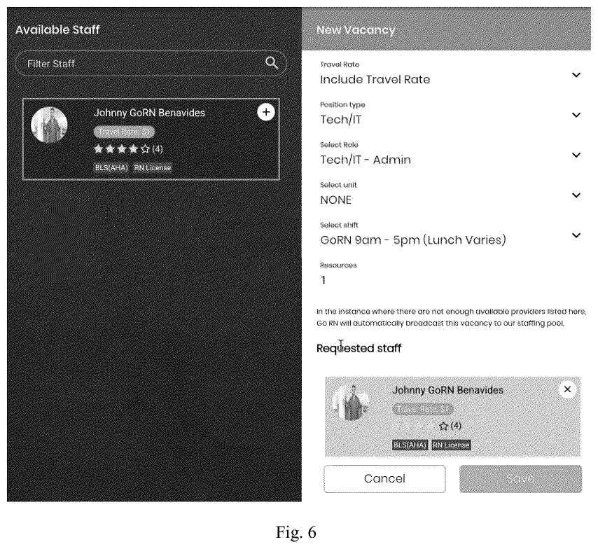

As discussed above and illustrated in FIG. 4, the provider 102 can use the provider portal to create an offer of shift availability before a demand for the shift even exists. The availability offer is stored in the server 112 and includes information such as the location of the shift he/she is available to work, role of the provider, and shift time that he/she is available. When the facility creates a vacancy request through facility portal using facility device 108, the server 112 will of course search through its database to find the provider most suitable to fulfill the vacancy based on location, credential, etc. However, if there is a suitable provider with availability offer already stored in the database, the server 112 can optionally show the provider right after the facility submitted the vacancy request. FIG. 6 is an example of what facility portal showing the provider with availability offer right next to the vacancy in question. As illustrated on the left side of FIG. 6, Johnny has already offered his availability for the shift in question. Thus, the facility can simply choose him to fulfill the vacancy. This saves the time to send invitation to potential providers and greatly reduce the possibility that there might not be anyone responding to the invitation.

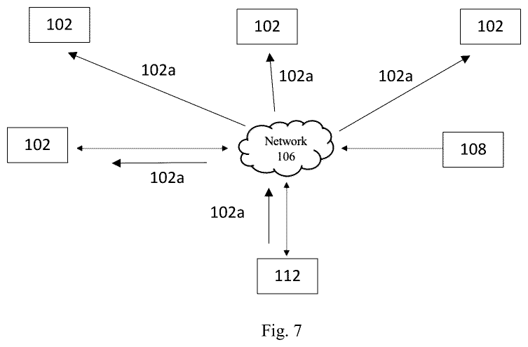

In one scenario, it is possible that no one responds or accepts the invitation to fulfill the shift vacancy. Thus, there is now a need to expand the scope of search for potential provider to fulfill the vacancy. Firstly, the server 112 may optionally determine an urgency parameter used as a factor in determining the number of outgoing invitation, wherein said urgency parameter is based at least in part on how close the shift in question is to the current time. For instance, the closer the current time is to the shift time, the more invitations need to be sent out and the urgency parameter should be adjusted to reflect the urgency. Then, a new list is created which includes a greater numbers of potential providers than the previous one. Primarily, the server 112 creates a bigger list of potential providers by expanding the geographical area centered around the facility. In some situation, the facility 108 may also be involved in the generation of a bigger list by adjusting parameters in the vacancy request 108a such as pay rate for the shift vacancy. However, the pay rate is preferably not changed to make sure that the providers' remuneration is consistent. If somehow there is still vacancy to be fulfilled after the latest round of invitation, then the server 112 can then create an ever bigger list the same way described above and repeat the process until the vacancy is either fulfilled or no longer existent, as illustrated in FIG. 7.

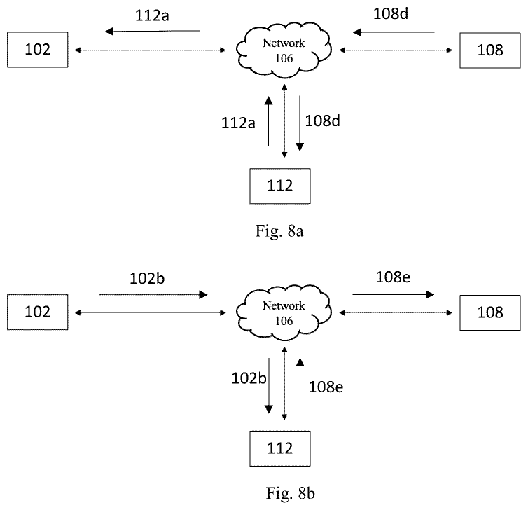

As discussed above, the facility 108 can make changes to the existing vacancy request such as pay rate, shift time, role and position for the shift through the facility portal. As illustrated in FIG. 8a, the facility 108 can make additions such as compliance or credential requirements 108d and send them to the server 112 for storage. The server 112 can then send compliance request 112a to the provider 102 and ask the provider 102 to verify or update the professional training, education, certification, licenses, or registration based on the compliance or credentials requirements set up by the facility. If the provide 102 fails to satisfy the requirements within certain time limit, he/she may be prevented, by the server 112, from appearing in the list of potential providers in the future. The provider 102 can satisfy the credential or compliance requirements by uploading documentations 102b such as copies of government registration and diploma, to the server 112 using the provider portal. The server 112 can then verify the documentation 102b uploaded and update the compliance/credential status of the provider. Optionally, the server 112 can give the facility 108 update 108e on the compliance status of the provider.

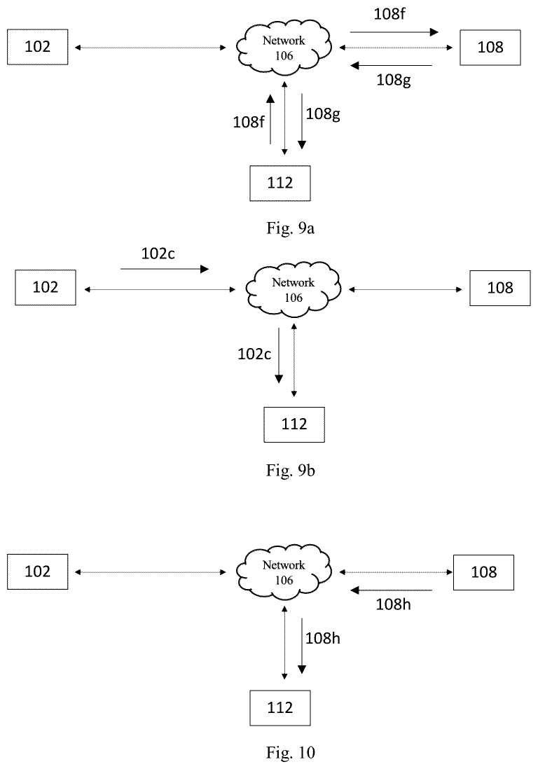

According to one embodiment of the present invention illustrated in FIG. 9a, the system of the present invention allows the facility to collect information regarding the performance of the providers. At the end of every shift, the server 112 will provide a questionnaire 108f to the facility through the facility portal. The questionnaire 108f may contain questions related to the performance of the provider during the shift such as types and number of mistakes made. The completed questionnaire 108g will then be sent to and stored in the database of the server 112 for future reference. Further, the information can be extracted from the completed questionnaire 108g and be used to construct a performance metrics model of the provider 102 in question. The resultant model is then used as an important factor during compilation of a list of potential providers to fulfill a recently occurring shift vacancy. For instance, the provider 102 with the best performance metrics model will likely to appear on top of the list of potential providers for facility's 108 review and vice versa. Thus, the performance metrics model can be a very useful tool for the facility 108 or server 112 to determine which provider 102 to invite to fulfill the currently available or future shift vacancy. Also, as illustrated in FIG. 9b, the server 112 may request shift confirmation 102c from the provider 102 both at the beginning and end of the shift in order to keep record of the begin and end time of shift for future references.

Also, continuity of care, idealized in the patient's experience of a ‘continuous caring relationship’ with an identified health care professional. Continuity of care is concerned with quality of care over time. It is the process by which the patient and his/her physician-led care team are cooperatively involved in ongoing health care management toward the shared goal of high quality, cost-effective medical care. It reduces fragmentation of care and thus improves patient safety and quality of care. Thus, it is advantageous to make the continuity of care an important factor when considering whether to invite certain provider to fulfill a shift vacancy at the same location. For instance, the provider who worked the longest at the location will likely appear on top of the list of potential providers for facility's review and vice versa.

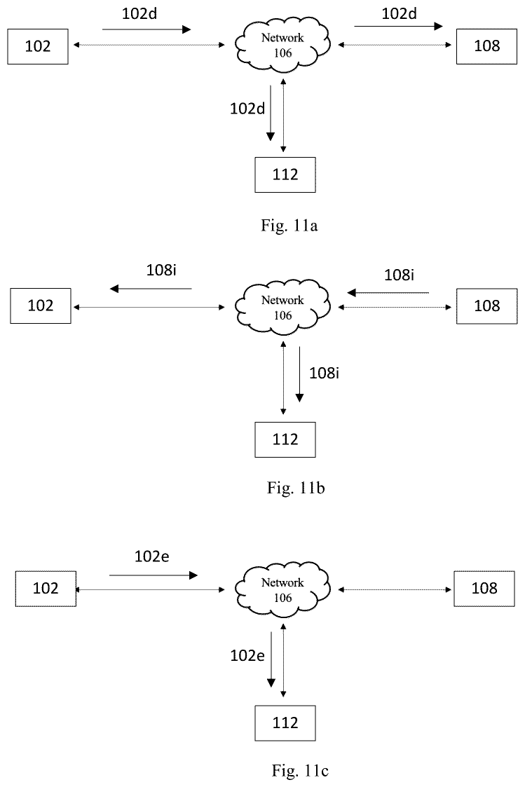

In the embodiment illustrated in FIG. 10, the system of the present invention allows the facility 108 to establish one or more criterion 108h that determines whether the provider 102 should no longer be working in the facility 108, at least temporarily. In other words, if the provider's 102 record shows that he/she has satisfied the exclusion criteria 108h set up by the facility 108, the server 112 will no longer take the provider 102 into consideration when compiling the list of potential provider to fulfill any vacancy in the future. The result is that the provider 102 will no longer be able to participate in the fulfillment of the shift vacancy for one specific facility. For instance, the provider may be excluded if he/she failed to satisfy general requirements such as professional training, education, certification, licenses, or registration for a given period of time. The provider can also be excluded for certain facility-specific requirements such as number of serious complaints or continuously poor performance review. There can be conditions according to which the provider may one day be reinstated again, depending on the provider's record. The criteria for exclusion can be based on the compliance requirements. For instance, the provider 102 may be reinstated after certain period of time (e.g., 3 months) or after supplying the requested documentations. In yet another embodiment, the server 112 can also include criteria 108h that determines whether the provider should be removed from the entire system. In other words, the entire provider portal and any information associated with the provider will be removed from the server 112.

In the embodiment illustrated in FIGS. 11a-c, the system also allows the provider to perform other functions such as requesting break. As illustrated in FIG. 11a-c, the provider 102 can send break request 102d through the network 106 to both the facility 108 for approval and the server 112 for recording. The facility 108 can approve the break request 102d by sending the break approval 108i through the network 106 to both the provider 102 for notification and the server 112 for recording. Also, the server 112 may request break confirmation 102e from the provider 102 both at the beginning and end of the break in order to keep record of the begin and end time of shift for future references.

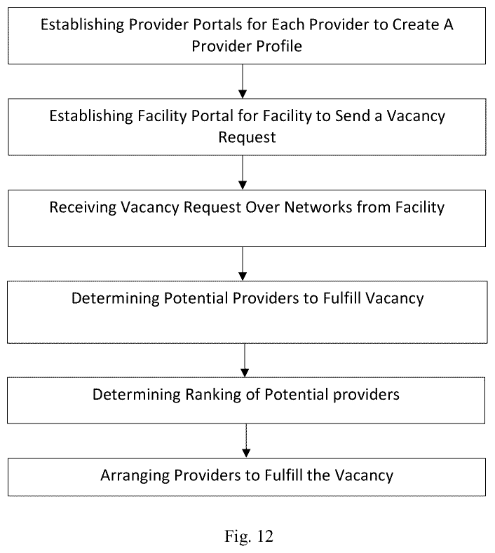

FIG. 12 is a flowchart of the method of managing a system of professional service providers and facilities that require professional services. The method includes step 201 of establishing provider portals for each provider to create a provider profile. The portal is a specially designed website that collect information from and distributes information to the professional service providers and facilities. For professional service providers, portals allow them to update their credentials for compliance, accept invitation to work shift, cancel shifts, give feedback at the end of shifts, etc. The method further includes step 202 of establishing facility portal for the facility to send vacancy requests. Further, portals allow facilities to set up credential or compliance requirements for providers to update and satisfy, creating vacancies of shifts to be fulfilled by providers, choose the providers on a recommendation list to fulfill the vacancies, etc. The activities mentioned above that can be done in portals by both providers and facilities will be discussed in more details below.

Once the portals for both providers and facilities are set up, the method can start step 203 of receiving vacancy requests over the network from facilities, using a server that has a database to store information at the portals. Once the vacancy request 106a is received, the method will perform step 204 of determining potential providers to fulfill vacancy. Once the server receives the vacancy request, it will review the provider information stored in the database to look for provider that can fulfill the vacancy, based on the information included in the vacancy request plus other information such as compliance status and credentials. The server then determines a list of potential providers to fulfill the vacancy based on information associated with each provider and the vacancy in question. Then the method will perform step 205 of determining ranking of potential providers on the list. The server ranks the potential providers based on criteria such as availabilities of the providers, credential, compliance status, etc. in order to find the best fit to fulfill the vacancy. Finally, the method performs step 206 of arranging providers to fulfill the vacancy. the server will send an invitations to potential providers for his/her acceptance to fulfill the vacancy. In another embodiment, the server may automatically send out invitation. For instance, the server may automatically send invitations to providers already declared their availabilities before the vacancy is created by the facility. If there is still vacancy to be fulfilled, then the server will create another list of potential providers for facility review and send out invitations automatically or upon facility approval, until all vacancies area fulfilled.

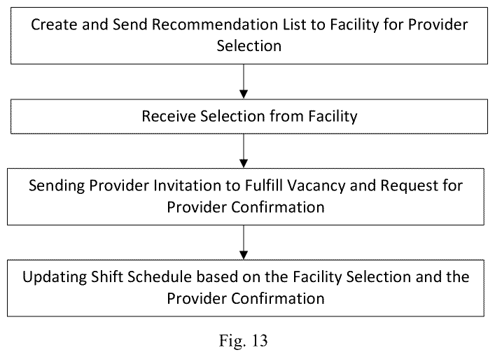

FIG. 13 illustrates an alternative embodiment of the method of arranging professional service providers to fulfill the vacancy at the facility. The method can further include step 301 of creating and sending a recommendation list to the facility for selection. The recommendation list is based on the ranking performed in step 205 on criteria such as availabilities of the providers, credential, compliance status, etc. in order to find the best fit to fulfill the vacancy. Generally, the provider 102 that is already available or has the best performance metrics model will likely to appear on top of the list of potential providers for facility's review and vice versa. The method then moves on the next step 302 of waiting for selection and approval from facility regarding which provider to invite to fulfill the vacancy. Once the selection is confirmed, the method then performs step 303 of sending invitations to potential providers to fulfill the vacancy and request for acceptance/confirmation from the provider. Once the server receives acceptance/confirmation from the provider, the method can execute step 304 of updating the shift schedule on the facility portal based on the provider selection by the facility and the acceptance/confirmation from the provider.

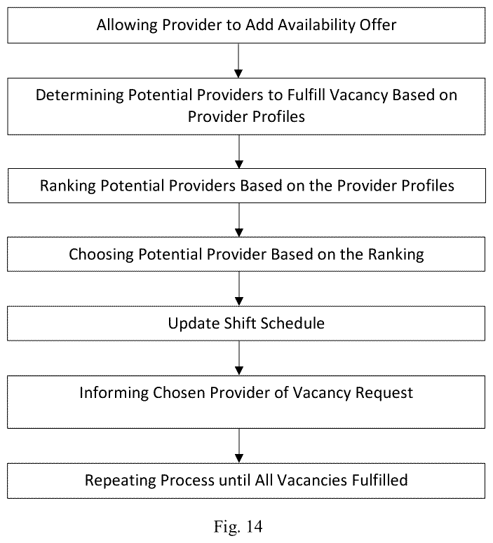

FIG. 14 illustrates an embodiment that allows provider to pre-offer his/her availability. Step 401 involves allowing a provider to add availability offer using his/her provider portal. The provider can supply information such as the location of the shift he/she is available to work, role of the provider, and shift time that he/she is available. Once the provider has confirmed his/her availability, the information will be sent from the provider device to the server for storage. The method will start step 402 when receiving a vacancy request from the facility. The server will check its database to see if there is any provider who has already offered available for the shift in question. If there is indeed one or more provider already available, the server 112 may automatically send out invitations to the providers for acceptance to fulfill the vacancy. Alternatively, the server may first send the recommendation list to the facility that includes the provider with availability, wait for the facility's choice/approval, and send invitation to the chosen provider. Step 403 of ranking potential providers, step 404 of choosing provider based on the ranking, and step 405 of updating shift schedule for facility's review have already been discussed above and will not be repeated here. Similarly, step 406 of informing provider of vacancy request using invitation has been discussed above and will also not be repeated here. However, if there is still vacancy to be fulfilled, the method will execute step 407 of repeating the vacancy fulfillment process from the beginning until there is no more vacancy.

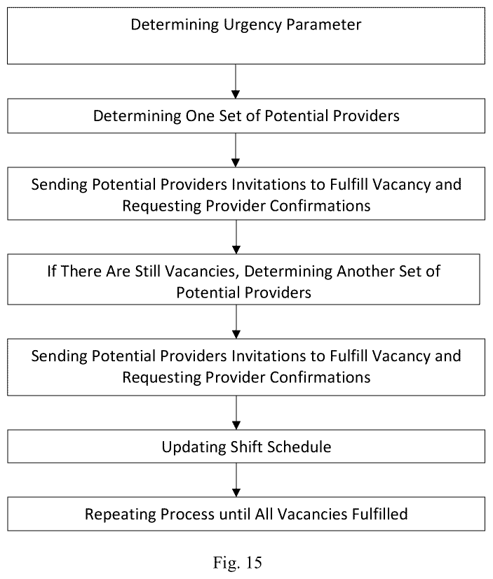

FIG. 15 illustrates the scenario that no one responds or accepts the invitation to fulfill the shift vacancy. Thus, there is now a need to expand the scope of search for potential provider to fulfill the vacancy. The method will execute step 501 of determining an urgency parameter used as a factor in determining the number of outgoing invitation, wherein said urgency parameter is based at least in part on how close the shift in question is to the current time. For instance, the closer the current time is to the shift time, the more invitations need to be sent out and the urgency parameter should be adjusted to reflect the urgency. Then, the method moves on to step 502 of determining list/set that includes a greater number of potent providers than the previous one. Primarily, the step 502 creates a bigger list of potential providers by expanding the geographical area centered around the facility. Then the rest of steps that involves sending out invitations to providers and updating shift schedule have been discussed above and will not be repeated here. Also, the method will continue executing the vacancy fulfillment process from the beginning until there is no more vacancy.

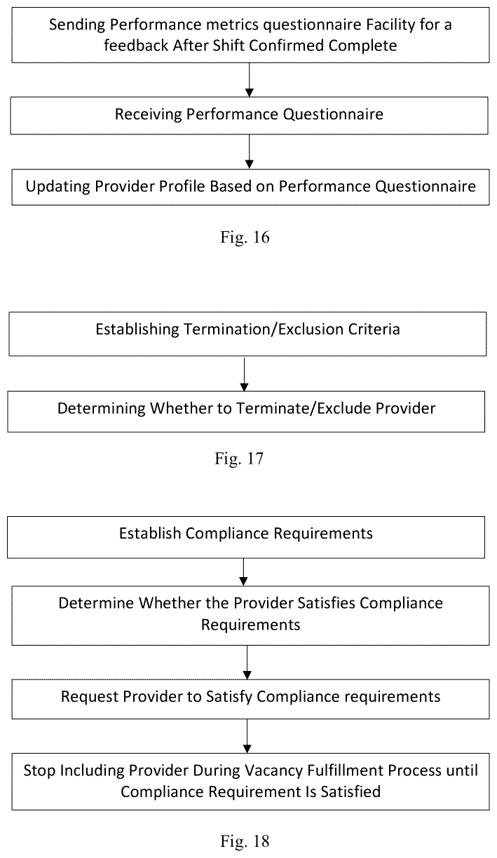

FIG. 16 illustrates another embodiment of the method that allows the facility to collect information regarding the performance of the providers. For step 601, at the end of every shift, the server will provide a questionnaire to the facility through the facility portal. The questionnaire may contain questions related to the performance of the provider during the shift such as types and number of mistakes made. In step 602, the completed questionnaire will then be sent to and stored in the database of the server for future reference. In step 603, the completed questionnaire will be used to update the profile of the provider in question. Furthermore, information is extracted from the completed questionnaire and used to construct a performance metrics model of the associated provider. The resultant model is then used as an important factor during compilation of a list of potential providers to fulfill a recently occurring shift vacancy. For instance, the provider with the best performance metrics model will likely to appear on top of the list of potential providers for facility's selection and vice versa. Thus, the performance metrics model can be a very useful tool for the facility or server to determine which provider to invite to fulfill the currently available or future shift vacancy.

FIG. 17 illustrates yet another embodiment of the method that includes step 701 of allowing the facility to establish one or more criterion that determines whether the provider should no longer be working in the facility, at least temporarily. If the provider's record shows that he/she has satisfied the exclusion criteria 108h set up by the facility, the method will execute step 702 so that the server will no longer take the provider into consideration when compiling the list of potential provider to fulfill any vacancy in the future. The result is that the provider will no longer be able to participate in the fulfillment of the shift vacancy for one specific facility. For instance, the provider may be excluded if he/she failed to satisfy general requirements such as professional training, education, certification, licenses, or registration for a given period of time. The provider can also be excluded for certain facility-specific requirements such as number of serious complaints or continuously poor performance review. There can be conditions according to which the provider may one day be reinstated again, depending on the provider's record. The criteria for exclusion can be based on the compliance requirements. For instance, the provider may be reinstated after certain period of time (e.g., 3 months) or after supplying the requested documentations. In yet another embodiment, the server can also include criteria that determines whether the provider should be removed from the entire system. In other words, the entire provider portal and any information associated with the provider will be removed from the server.

FIG. 18 illustrates yet another embodiment of the method that includes step 801 of allowing the facility to make additions such as compliance or credential requirements and send them to the server for storage. The method then can executive step 802 of using the server to check the provider portal to determine whether the provide satisfies the compliance requirements. If not, then the method will execute step 803 of sending compliance request to the provider and ask the provider to verify or update the professional training, education, certification, licenses, or registration based on the compliance or credentials requirements set up by the facility. If it is determined that the provider fails to satisfy the compliance requirements for an unreasonable period of time, the method can execute step 804 of not including the provider in the list of potential provider to fulfill any future vacancy, until the provider has finally satisfied the compliance requirements. The provider can satisfy the credential or compliance requirements by uploading documentations such as copies of government registration and diploma, to the server using the provider portal. The server can then verify the documentation uploaded and update the compliance/credential status of the provider.

Reference throughout this specification to “one embodiment,” “an embodiment,” “one example,” or “an example” means that a particular feature, structure, or characteristic described in connection with the embodiment or example is included in at least one embodiment of the present disclosure. Thus, appearances of the phrases “in one embodiment,” “in an embodiment,” “one example,” or “an example” in various places throughout this specification are not necessarily all referring to the same embodiment or example. Furthermore, the particular features, structures, databases, or characteristics may be combined in any suitable combinations and/or sub-combinations in one or more embodiments or examples. In addition, it should be appreciated that the figures provided herewith are for explanation purposes to persons ordinarily skilled in the art and that the drawings are not necessarily drawn to scale.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims. It is also understood that other embodiments of this invention may be practiced in the absence of an element/step not specifically disclosed herein.

1. A computing system for managing a network-based service, the computing system comprising:
one or more processors; and
one or more memory resources storing instructions that, when executed by the one or more processors, cause the computing system to:
establish a provider portal for a plurality of providers to each create a provider profile;
establish a facility portal for at least one facility to send a vacancy request to fulfill one or more vacancies;
receive, over one or more networks, the vacancy request from the facility;
determine a set of potential providers, to fulfill the vacancies, based on the provider profiles including availabilities, a compliance requirement, and the vacancy request, wherein the compliance requirement includes a government compliance requirement and a facility compliance requirement;
determine a ranking of the set of the providers based on the provider profiles and the vacancy request; and
arrange the providers to fulfill the vacancies based at least in part on the ranking and the provider profiles until all vacancies have been fulfilled.

2. The computer system of claim 1, wherein the executed set of instructions further causes the computer system to:
send a first recommendation list based on the ranking, over one or more networks, to the facility for a facility confirmation;
receive the facility confirmation from the facility; and
sending one or more providers, based on the facility confirmation, invitations regarding the vacancy request and request for provider confirmations; and
updating a shift schedule based on the facility confirmations and the provider confirmations.

3. The computer system of claim 2, wherein when the provider cancels the provider confirmation, the executed set of instructions further causes the computer system to:
determine a new set of potential providers based on the provider profiles, the compliance requirement, and the vacancy request;
determine a new ranking of the set of the providers based on the provider profiles and the vacancy request;
send a second recommendation list based on the new ranking, to the facility for a new facility confirmation;
send one or more provider, based on the new facility confirmation, invitations regarding the vacancy request and request for provider confirmations; and
update the shift schedule based on the provider confirmations.

4. The computer system of claim 1, wherein the executed set of instructions further causes the computer system to:
allow one or more providers to add an availability offer to the respective provider profile, wherein the availability offer includes a shift time, a pay rate, a role, and a preferred area;
determine the set of potential providers based on the respective provider profile with the availability offer;
determine the ranking of the set of providers based on the provider profile with the availability offer;
choose one or more providers based on the ranking to fulfill the vacancies;
update a shift schedule based on the chosen providers and the respective provide profiles;
inform the chosen providers of the vacancy request and the shift schedule; and
repeat the process until all the vacancies have been fulfilled.

5. The computer system of claim 4, wherein when the provider cancels the availability offer after the shift schedule has been updated, the executed set of instructions further causes the computer system to:
determine a new set of potential providers based on the associated provider profiles with the availability offers;
determine a new ranking of the new set of providers based on the provider profile with the availability offer;
choose one or more providers based on the ranking to fulfill the vacancies;
update the shift schedule based on the chosen providers; and
inform the chosen provider of the vacancy request and the shift schedule.

6. The computer system of claim 4, wherein if there are no more providers with the availability offer to fulfill the vacancies, the executed set of instructions further causes the computer system to:
determine an urgency parameter based at least in part on a current time and a time of the vacancies;
determine a first set of potential providers based on the provider profiles, the compliance requirement, the urgency parameter, and a location of the facility;
send the providers within the first set invitations to fulfill the vacancy and request for provider confirmations;
if there are still vacancies to be fulfilled after sending invitations to the first set of providers, determine a second set of potential providers based on the associated provider profiles, the compliance requirement, the urgency parameter, and the location of the facility, wherein the second set includes more potential providers than the first set;
send the providers within the second set invitations to fulfill the vacancy and request for provider confirmations;
update a shift schedule based on the provider confirmation; and
continue until all vacancies have been fulfilled.

7. The computer system of claim 1, wherein the facility can update the vacancy request at the facility portal, the providers can update their provider profiles at the provider portal, the provider profile includes one or more certification, one or more licenses, one or more government documentations, a pay rate, an aggregate performance rating, and a residence area.

8. The computer system of claim 1, wherein the executed set of instructions further causes the computer system to:
send a performance metrics questionnaire, after a shift is confirm complete, to the facility for a feedback regarding the provider's performance during the shift;
receive the performance questionnaire; and
update the provider profile of the provider based on the performance questionnaire.

9. The computer system of claim 1, wherein the executed set of instructions further causes the computer system to:
establish a termination criterion based on the compliance requirement and a facility requirement from the facility; and
determine, based on the provider profile and the termination criterion, whether to terminate one of the providers in the provider portal.

10. The computer system of claim 1, wherein the executed set of instructions further causes the computer system to:
determine whether the provider is compliant with regards to the compliance requirement based on the provider profile;
request the provider to update the provider profile to satisfy the compliance requirement; and
stop including the provider during the vacancy fulfillment process until the compliance requirement is satisfied.

11. A computer-implemented method for managing a network-based service, comprising:
establishing a provider portal for a plurality of providers to each create a provider profile;
establishing a facility portal for at least one facility to send a vacancy request to fulfill one or more vacancies;
receiving, over one or more networks, the vacancy request from the facility;
determining a set of potential providers, to fulfill the vacancies, based on the provider profiles including availabilities, a compliance requirement, and the vacancy request,
wherein the compliance requirement includes a government compliance requirement and a facility compliance requirement;
determining a ranking of the set of the providers based on the provider profiles and the vacancy request; and
arranging the providers to fulfill the vacancies based at least in part on the ranking and the provider profiles until all vacancies have been fulfilled.

12. The computer-implemented method of claim 11, further comprising:
send a first recommendation list based on the ranking, over one or more networks, to the facility for a facility confirmation;
receive the facility confirmation from the facility; and
sending one or more providers, based on the facility confirmation, invitations regarding the vacancy request and request for provider confirmations; and
updating a shift schedule based on the facility confirmations and the provider confirmations.

13. The computer-implemented method of claim 12, when the provider cancels the provider confirmation, further comprising:
determining a new set of potential providers based on the provider profiles, the compliance requirement, and the vacancy request;
determining a new ranking of the set of the providers based on the provider profiles and the vacancy request;
sending a second recommendation list based on the new ranking, to the facility for a new facility confirmation;
sending one or more provider, based on the new facility confirmation, invitations regarding the vacancy request and request for provider confirmations; and
updating the shift schedule based on the provider confirmations.

14. The computer-implemented method of claim 11, further comprising:
allowing one or more providers to add an availability offer to the respective provider profile, wherein the availability offer includes a shift time, a pay rate, a role, and a preferred area;
determining the set of potential providers based on the respective provider profile with the availability offer;
determining the ranking of the set of providers based on the provider profile with the availability offer;
choosing one or more providers based on the ranking to fulfill the vacancies;
update a shift schedule based on the chosen providers and the respective provide profiles;
informing the chosen providers of the vacancy request and the shift schedule; and
repeating the process until all the vacancies have been fulfilled.

15. The computer-implemented method of claim 14, when the provider cancels the availability offer after the shift schedule has been updated, further comprising:
determining a new set of potential providers based on the associated provider profiles with the availability offers;
determining a new ranking of the new set of providers based on the provider profile with the availability offer;
choosing one or more providers based on the ranking to fulfill the vacancies;
updating the shift schedule based on the chosen providers; and
informing the chosen provider of the vacancy request and the shift schedule.

16. The computer-implemented method of claim 14, if there are no more providers with the availability offer to fulfill the vacancies, further comprising:
determining an urgency parameter based at least in part on a current time and a time of the vacancies;
determining a first set of potential providers based on the provider profiles, the compliance requirement, the urgency parameter, and a location of the facility;
sending the providers within the first set invitations to fulfill the vacancy and request for provider confirmations;
if there are still vacancies to be fulfilled after sending invitations to the first set of providers, determining a second set of potential providers based on the associated provider profiles, the compliance requirement, the urgency parameter, and the location of the facility, wherein the second set includes more potential providers than the first set;
sending the providers within the second set invitations to fulfill the vacancy and request for provider confirmations;
updating a shift schedule based on the provider confirmation; and
continuing until all vacancies have been fulfilled.

17. The computer-implemented method of claim 11, further comprising:
allowing the facility to update the vacancy request at the facility portal; and
allowing the providers to update provider profiles at the provider portal, wherein the provider profile includes one or more certification, one or more licenses, one or more government documentations, a pay rate, an aggregate performance rating, and a residence area.

18. The computer-implemented method of claim 11, further comprising:
sending a performance metrics questionnaire, after a shift is confirm complete, to the facility for a feedback regarding the provider's performance during the shift;
receiving the performance questionnaire; and
updating the provider profile of the provider based on the performance questionnaire.

19. The computer-implemented method of claim 11, further comprising:
establishing a termination criterion based on the compliance requirement and a facility requirement from the facility; and
determining, based on the provider profile and the termination criterion, whether to terminate one of the providers in the provider portal.

20. The computer-implemented method of claim 11, further comprising:
determine whether the provider is compliant with regards to the compliance requirement based on the provider profile;
request the provider to update the provider profile to satisfy the compliance requirement; and
stop including the provider during the vacancy fulfillment process until the compliance requirement is satisfied.
